(19) United States
(12) Patent Application Publication
(10) Pub. No.: US 2016/0213527 A1
(43) Pub. Date: Jul. 28, 2016

(54) Refastenable Absorbent Article

(71) Applicant: The Procter & Gamble Company, Cincinnati, OH (US)

(72) Inventors: Gary Dean LaVON, Liberty Township, OH (US); Bret Darren SEITZ, West Chester, OH (US); Masaharu NISHIKAWA, Cincinnati, OH (US); Bryan Keith WAYE, Mason, OH (US); Thomas Ludwig WOSCHNIK, Euskirchen (DE); Yoichiro YAMAMOTO, Koeln (DE); Thomas TOMBUELT, Nettersheim (DE); Hans Adolf JACKELS, Mechernich (DE)

(21) Appl. No.: 15/090,721

(22) Filed: Apr. 5, 2016

Related U.S. Application Data
(63) Continuation of application No. 13/010,083, filed on Jan. 20, 2011.

(51) Int. Cl. A61F 13/49; A61F 13/56

(52) U.S. Cl. CPC A61F 13/49015; A61F 13/5655

(57) ABSTRACT

Absorbent articles such as disposable diapers, incontinent briefs, diaper holders, training pants and the like, having elastomeric side panels and a fastening system that provides the user with different options as to how the absorbent article may be fitted to and removed from the wearer. The absorbent articles allow the wearer to choose between conventional and pull-on diaper configurations, or combinations thereof, and properly and comfortably fit a large range of wearer sizes. Further, the absorbent articles can be pulled on and/or off as a pant.

### CROSS REFERENCE TO RELATED APPLICATION

This application is a continuation of, and claims priority under 35 U.S.C. §120 to, U.S. patent application Ser. No. 13/010,083, filed on Jan. 20, 2011, which claims the benefit, under 35 USC §119(e), of U.S. Provisional Patent Application Ser. No. 61/296,693, filed on Jan. 20, 2010, the entire disclosures of which are fully incorporated by reference herein.

### FIELD

The present disclosure generally relates to absorbent articles, and more particularly relates to absorbent articles having one or more elastomeric panels and a fastening system that provides the user with different options as to how the diaper will be fitted to a wearer.

### BACKGROUND

Infants and other incontinent individuals wear absorbent articles such as diapers to receive and contain urine and other body exudates. Absorbent articles function both to contain discharged materials and to isolate the materials from the body of the wearer and from the wearer's garments and bed clothing. Many of the absorbent articles on the market today, however, are capable of fitting only a small range of wearer sizes and therefore, the consumer must continually monitor the size of the wearer to determine which diaper will comfortably and effectively fit the wearer. Further, most commercially available diapers are limited in that they can only effectively be used in one configuration, such as a “taped” diaper or a “pull-on” diaper, without any of the benefits attributable to any of the other diaper types. Thus, the consumer must purchase different diapers depending on the desired characteristics for the intended use. Further, in attempting to fit a large range of wearers, previous diaper designs (both conventional taped diapers and pull-on diapers) also have generally been loose and ineffective containing the waste of small wearers while being extremely tight, uncomfortable and unable to withstand the dynamic forces produced by large wearers. Many pull-on designs lacked any means for checking the diaper for soiling without removal of the diaper and often the wearer's clothing. Likewise, pull-on designs generally lack any means for removing a soiled diaper without having to tear one or more elements (generally seams) of the diaper which can renders the diaper useless if checked for soiling. Diaper designs have been unable to provide a diaper that effectively and comfortably provides at least two ways in which the diaper may be constructed to fit to the wearer. What is needed is an improvement over the foregoing.

### BRIEF DESCRIPTION OF THE DRAWINGS

The above-mentioned and other features and advantages of the present disclosure, and the manner of attaining them, will become more apparent and the disclosure itself will be better understood by reference to the following description of non-limiting embodiments of the disclosure taken in conjunction with the accompanying drawings.

In the drawing and in the written description, lowercase letters appended to reference numerals indicate generally symmetric elements, e.g., left and right symmetric elements may be respectively identified by the reference numerals 1a and 1b. A reference numeral without an appended lowercase letter identifies all of the elements to which that particular reference numeral applies.

FIG. 1 is a plan view of a four side panel embodiment of an absorbent article in accordance with one non-limiting embodiment with the outer surface of the absorbent article facing the viewer.

FIG. 2 is a plan view of a two side panel embodiment of an absorbent article in accordance with one non-limiting embodiment with the outer surface of the absorbent article facing the viewer.

FIG. 3 is a plan view of a four side panel embodiment of an absorbent article in accordance with one non-limiting embodiment with the outer surface of the absorbent article facing the viewer.

FIGS. 4-5 are perspective views of absorbent articles in pull-on configurations in accordance with non-limiting embodiments.

FIG. 6A-6E are partial views of a side panel in accordance with various non-limiting embodiments.

FIG. 7 is a partial view of an absorbent article comprising releasable bonds in accordance with one non-limiting embodiment.

FIG. 8 is a partial view of an absorbent article comprising a force distribution portion in accordance with one non-limiting embodiment.

FIG. 9 is a partial view of an absorbent article in accordance with one non-limiting embodiment.

FIG. 10 is a partial view of an absorbent article comprising an adjustable waist hoop circumference in accordance with one non-limiting embodiment.

FIGS. 11A-11C are a progression of an absorbent article comprising a permanent seam and a refastenable flap in accordance with one non-limiting embodiment

FIGS. 12A-12C are partial views of various embodiments of side panels and the engaging components joined thereto.

FIGS. 13A-13C are partial views of various embodiments of side panels with various bond patterns joining engaging components thereto.

FIG. 14 is a schematic view of equipment used to make the absorbent articles of the present disclosure in accordance with various non-limiting embodiments.

### DETAILED DESCRIPTION

Various non-limiting embodiments of the present disclosure will now be described to provide an overall understanding of the principles of the structure, function, manufacture, and use of the apparatuses and methods disclosed herein. One or more examples of these non-limiting embodiments are illustrated in the accompanying drawings. Those of ordinary skill in the art will understand that the apparatuses and methods specifically described herein and illustrated in the accompanying drawings are non-limiting example embodiments and that the scope of the various non-limiting embodiments of the present disclosure are defined solely by the claims. The features illustrated or described in connection with one non-limiting embodiment may be combined with the features of other non-limiting embodiments. Such modifications and variations are intended to be included within the scope of the present disclosure.

In this description, the following terms have the following meanings:

The term “absorbent article” refers to a device that is placed against or in proximity to a body of a wearer to absorb and contain the various exudates discharged from the body. Example absorbent articles may comprise diapers, training pants, pull-on pant-type diapers (i.e., a diaper having a pre-formed waist opening and leg openings, such as illustrated in U.S. Pat. No. 6,120,487), refastenable diapers, incontinence briefs and undergarments, diaper holders and liners, feminine hygiene garments and related articles.

The term “disposable” refers to the nature of absorbent articles that generally are not intended to be laundered or otherwise restored or reused as an absorbent article, i.e., they are intended to be discarded after a single use and to be recycled, composted or otherwise disposed of in an environmentally compatible manner.

The term “diaper” refers to an absorbent article generally worn by infants and incontinent persons that is worn about the lower torso of the wearer. It should be understood, however, that the present disclosure is also applicable to other absorbent articles such as incontinence briefs, incontinence undergarments, diaper holders and liners, feminine hygiene garments, training pants, and the like.

The term “joined” encompasses configurations whereby an element is directly secured to the other element by affixing the element directly to the other element, and configurations whereby the element is indirectly secured to the other element by affixing the element to intermediate member(s) which in turn are affixed to the other element.

The term “stretchable” refers to materials that are extensible when forces are applied to the material, and offer some resistance to extension.

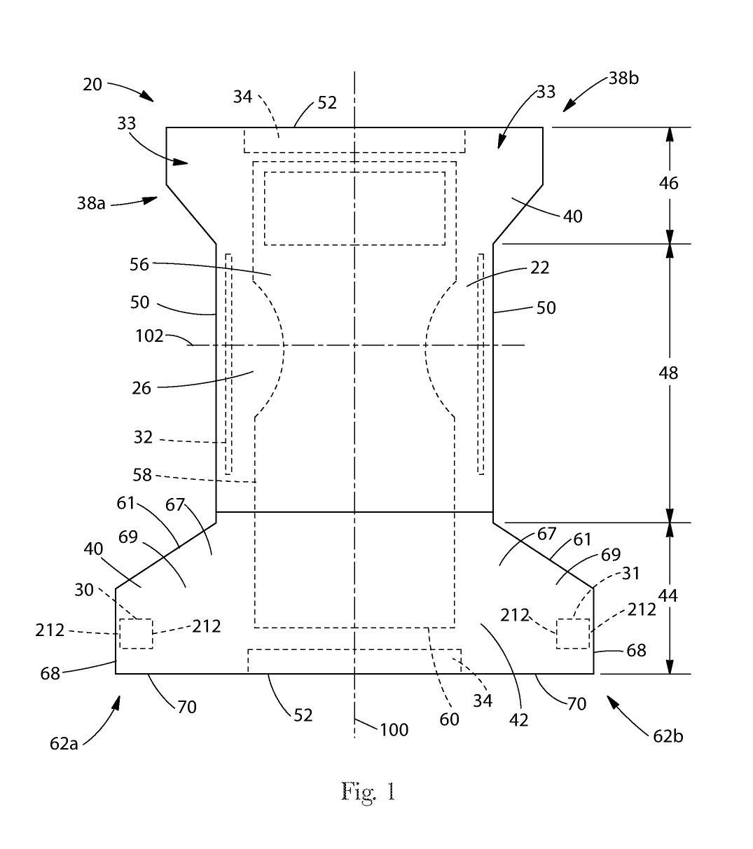

A “unitary” absorbent article refers to absorbent articles which are formed of separate parts united together to form a coordinated entity so that they do not require separate manipulative parts like a separate holder and liner. An embodiment of an absorbent article is the disposable absorbent article, diaper 20, shown in FIG. 1.

The consumer has a number of different basic diaper designs to choose from depending on the desired options, comfort and cost, including conventional diapers, belted diapers, and “pull-on” type diapers or training pants. Conventional diaper designs, such as taped diapers, are generally a relatively inexpensive type of absorbent article to produce and are generally acceptable for use on babies and persons who are sick or otherwise confined to a bed. A conventional diaper is fitted to the wearer by first placing a portion of the diaper under the wearer (generally, the back portion of the diaper is placed under the buttocks and rear waist of the wearer) and then pulling the remainder of the diaper through the wearer's legs. The rear portion of the diaper is then attached to the front portion of the diaper on each side of the wearer. However, such conventional configurations tend to be very difficult to use when the wearer refuses to remain still throughout the period of application. Further, adult wearers and children in their toilet training stage often find the conventional type absorbent articles difficult to put on themselves without assistance.

The “pull-on” design absorbent article is often used in training pants and incontinence briefs. The “pull-on” design allows the wearer to pull the absorbent article on as pants and does not require any of the fastening steps of the conventional or belted type absorbent article designs. Although this feature is desirable for many adult users and children in their toilet training stage, the “pull-on” design may be impractical for many users, especially those bed ridden and small children unable to dress themselves. Pull-on absorbent articles generally lack any features that allow the diaper to be put on, removed or checked for soiling without removal of the diaper and the wearer's outer clothing. Further, such articles often lack features that allow for convenient, sanitary disposal of the article.

In various embodiments, the present disclosure generally combines the benefits of a conventional diaper with those of a “pull-on” type diaper. Further, the diaper of the present disclosure may comfortably and effectively fit a large range of wearer sizes. These unique characteristics are provided by the inventive coordination of special panels and fastening elements. The side panels and fastening elements work together to resolve the problems encountered in earlier attempts to provide some of the features of the present disclosure. For example, various embodiments can be fitted to a wide range of wearers in conventional and pull-on configurations, and provide excellent containment and comfort characteristics due to the ability of the side panels to provide a snug fit when fitted to small wearers as well as a snug comfortable fit to much larger wearers. The refastenable fastening system provides a strong closure that can withstand the dynamic forces created by the motions of the wearer; the forces generally being different depending on the size and age of the wearer.

Further, in various embodiments, structural design of the diaper provides the user with the opportunity to use the diaper as a pull-on. The side panels provide the necessary stretch to effectively accommodate a large range of wearers as well as the stretch needed to provide for easy application as a pull-on diaper. Again, the fastening system complements the side panels to provide a strong closure, yet still provides a means for removing the pull-on diaper by opening the closures rather than pulling the diaper down over the hips of the wearer. This configuration makes the removal of a soiled diaper much quicker and cleaner without the need to remove the wearer's clothing, such as shoes, for example. Furthermore, the refastenable fastening system provides a pull-on user with the ability to check for soiling of the diaper by opening one or more of the closures rather than removing the diaper completely. Also, the absorbent article of the present invention can easily be configured with a disposal feature (often the fastening system) which provides a convenient, sanitary means for disposing of the soiled article.

Thus, the present disclosure, in part, teaches an absorbent article having a refastenable fastening system and stretchable side panels designed to allow the absorbent article to be fitted to the wearer in both conventional and “pull-on” configurations. Additionally, in various embodiments, an absorbent article is disclosed that will effectively and comfortably fit a large range of wearer sizes in either a conventional or a pull-on configuration.

FIG. 1 is a plan view of one embodiment of a diaper 20 in its flat-out, uncontracted state (i.e., with elastic induced contraction pulled out) and with the portion of the diaper 20 which faces away from the wearer, the garment facing surface 40, facing the viewer. As shown in FIG. 1, the diaper 20 may comprise a containment assembly 22 comprising a liquid pervious topsheet 24; a liquid impervious backsheet 26 joined to the topsheet; and an absorbent core 28 positioned between the topsheet 24 and the backsheet 26. The diaper 20 may further comprise elasticized leg cuffs 32; waist features 34; and a fastening system 200 comprising a first closure member 30, a second closure member 31, and at least one third closure member 33.

The diaper 20 is shown in FIGS. 1-3 to have a garment facing surface 40 (facing the viewer), a body facing surface 42 opposed to the garment facing surface 40, a rear waist region 44, a front waist region 46 longitudinally opposed to the rear waist region 44, a crotch region 48 positioned between the rear waist region 44 and the front waist region 46, and a periphery which is defined by the outer perimeter or edges of the diaper 20 in which the longitudinally extending laterally opposing side edges are designated 50 and the laterally extending longitudinally opposing end edges are designated 52. The body facing surface 42 of the diaper 20 comprises that portion of the diaper 20 which is positioned adjacent to the wearer's body during use (i.e., the body facing surface 42 generally is formed by at least a portion of the topsheet 24 and other components joined to the topsheet 24). The garment facing surface 40 comprises that portion of the diaper 20 which is positioned away from the wearer's body (i.e., the garment facing surface 40 is generally formed by at least a portion of the backsheet 26 and other components joined to the backsheet 26). The rear waist region 44 and the front waist region 46 extend from the end edges 52 of the periphery to the crotch region 48.

The diaper 20 also has two centerlines, a longitudinal centerline 100 and a transverse centerline 102. The term “longitudinal”, as used herein, refers to the direction that is associated with the length of the diaper in the machine direction. The longitudinal centerline 100, or longitudinal axis, generally extends from the midpoint of the edge 52 of the diaper 20 in the front waist region 46 to the midpoint of the waist edge 52 of the rear waist region 44 of the diaper 20. The terms “transverse” and “lateral”, as used herein, are interchangeable and refer to the direction that is associated with the width of the diaper 20 in the cross-machine direction. The lateral centerline 102, or lateral axis, generally extends from the midpoint of one of the side edges 50 of the diaper to the midpoint of the laterally opposing side edge 50 of the diaper 20.

The containment assembly 22 of the diaper 20 is shown in FIG. 1 as comprising the central portion (chassis) of the diaper 20. The containment assembly 22 comprises at least an absorbent core 28 and may comprise inner and outer covering layers comprising the topsheet 24 and the backsheet 26, respectively. For unitary absorbent articles, the containment assembly 22 may comprise the central portion of the diaper which may be joined with other features, such as side panels, for example, to form the composite diaper structure. Thus, the containment assembly 22 for the diaper 20 generally comprises the topsheet 24, the backsheet 26, and the absorbent core 28.

In one embodiment, the containment assembly 22 comprises a topsheet 24 and a backsheet 26 which have length and width dimensions generally larger than those of the absorbent core 28. The topsheet 24 and the backsheet 26 extend beyond the edges of the absorbent core 28 to thereby form a portion of the periphery of the diaper 20. While the topsheet 24, the backsheet 26, and the absorbent core 28 may be assembled in a variety of known configurations, exemplary containment assembly configurations are described generally in U.S. Pat. No. 3,860,003, entitled “Contractible Side Portions for Disposable Diaper” which issued to Kenneth B. Buell on Jan. 14, 1975; and U.S. Pat. No. 5,151,092, entitled “Absorbent Article With Dynamic Elastic Waist Feature Having A Predisposed Resilient Flexural Hinge” which issued to Kenneth B. Buell et al., on Sep. 29, 1992; each of which is incorporated herein by reference.

The absorbent core 28 may be any absorbent member which is generally compressible, conformable, non-irritating to the wearer's skin, and capable of absorbing and retaining liquids such as urine and other certain body exudates. As shown in FIGS. 1-3, the absorbent core 28 has a garment-facing side 54, a body-facing side 56, a pair of side edges designated 58, and a pair of waist edges designated 60. One embodiment of the diaper 20 has an asymmetric, modified T-shaped absorbent core 28 having ears in the front waist region 46 but a generally rectangular shape in the rear waist region 44. However, the absorbent core 28 may be manufactured in a wide variety of sizes and shapes (e.g., rectangular, hourglass, asymmetric, etc.) and from a wide variety of liquid-absorbent materials commonly used in disposable diapers and other absorbent articles such as comminuted wood pulp which is generally referred to as airfelt. Examples of other suitable absorbent materials include creped cellulose wadding; meltblown polymers including coform; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials.

The configuration and construction of the absorbent core 28 may vary (e.g., the absorbent core may have varying caliper zones, a hydrophilic gradient, a superabsorbent gradient, or lower average density and lower average basis weight acquisition zones; or may comprise one or more layers or structures). Further, the size and absorbent capacity of the absorbent core 28 may also be varied to accommodate wearers ranging from infants through adults. The total absorbent capacity of the absorbent core 28 should be compatible with the design loading and the intended use of the diaper 20. Absorbent structures for use as the absorbent core 28 of the present disclosure that have generally achieved wide acceptance and commercial success are described in U.S. Pat. No. 4,610,678, entitled “High-Density Absorbent Structures” issued to Weisman et al. on Sep. 9, 1986; U.S. Pat. No. 4,673,402, entitled “Absorbent Articles With Dual-Layered Cores” issued to Weisman et al. on Jun. 16, 1987; U.S. Pat. No. 4,888,231, entitled “Absorbent Core Having A Dusting Layer” issued to Angstadt on Dec. 19, 1989; and U.S. Pat. No. 4,834,735, entitled “High Density Absorbent Members Having Lower Density and Lower Basis Weight Acquisition Zones” issued to Alemany et al. on May 30, 1989, each of which is incorporated herein by reference. The absorbent core may further comprise a dual core system containing an acquisition/distribution component formed in part of chemically stiffened fibers positioned over an absorbent storage core, as detailed in U.S. Pat. No. 5,234,423, entitled “Absorbent Article With Elastic Waist Feature and Enhanced Absorbency” issued to Alemany et al., on Aug. 10, 1993; and in U.S. Pat. No. 5,147,345, entitled “High Efficiency Absorbent Articles For Incontinence Management” issued to Young, et al., on Sep. 15, 1992, each of which is incorporated herein by reference.

The backsheet 26 is positioned adjacent the garment-facing surface 54 of the absorbent core 28 and may be joined thereto by attachment means such as those well known in the art. For example, the backsheet 26 may be secured to the absorbent core 28 by a uniform continuous layer of adhesive, a patterned layer of adhesive, or an array of separate lines, spirals, or spots of adhesive. Adhesives which have been found to be satisfactory are manufactured by H. B. Fuller Company of St. Paul, Minn., marketed as HL-1258, and Findley Adhesives Corporation of Wauwatosa, Wis., marketed as Findley 2031. An example of a suitable attachment technique comprising an open pattern network of filaments of adhesive is disclosed in U.S. Pat. No. 4,573,986, entitled “Disposable Waste-Containment Garment” issued to Minetola et al. on Mar. 4, 1986. Another suitable attachment means comprising several lines of adhesive filaments swirled into a spiral pattern is illustrated by the apparatus and methods shown in U.S. Pat. No. 3,911,173, entitled “Adhesive process” issued to Sprague, Jr. on Oct. 7, 1975; U.S. Pat. No. 4,785,996, entitled “Adhesive spray gun and nozzle attachment” issued to Ziecker, et al. on Nov. 22, 1978; and U.S. Pat. No. 4,842,666, entitled “Process for the Permanent Joining of Stretchable Threadlike or Small Ribbonlike Elastic Elements to a Flat Substrate, as well as use thereof for Producing Frilled Sections of Film or Foil Strip” issued to Werenicz on Jun. 27, 1989. Each of these patents are incorporated herein by reference. Alternatively, the attachment means may comprise, for example, heat bonds, pressure bonds, ultrasonic bonds, dynamic mechanical bonds, or any other suitable attachment means or combinations of these attachment means as are known in the art. In some embodiments however, the absorbent core is not joined to the backsheet 26, the topsheet 24, or both in order to provide desired performance, such as greater extensibility throughout the diaper 20, for example.

In one embodiment, the backsheet 26 is impervious to liquids (e.g., urine) and is manufactured from a thin plastic film, although other flexible liquid impervious materials may also be used. As used herein, the term “flexible” refers to materials which are compliant and will readily conform to the general shape and contours of the human body. The backsheet 26 prevents the exudates absorbed and contained in the absorbent core 28 from wetting articles which contact the diaper 20 such as bedsheets and undergarments. However, the backsheet 26 is preferably breathable so as to permit moisture vapor to escape from the absorbent core 28 while still preventing exudates in liquid form from passing through the backsheet 26. The backsheet 26 may comprise woven or nonwoven materials, polymeric films such as thermoplastic films of polyethylene or polypropylene, or combinations thereof. A suitable backsheet 26 is a thermoplastic film having a thickness of from about 0.012 mm (0.5 mil) to about 0.051 mm (2.0 mils) joined with a nonwoven covering comprising natural or synthetic fibers. If the backsheet 26 is a film, it may be embossed and/or matte finished to provide a more clothlike appearance.

In one embodiment, at least a portion of the backsheet 26 is subjected to mechanical stretching in order to provide both a “zero strain” stretch laminate that forms the portion of the backsheet 26 coinciding with the waist feature 34. The backsheet 26 can be prestrained by directing the backsheet through an incremental mechanical stretching system similar to the operation described with respect to the formation of the “zero strain” stretch laminate backsheet and elasticized ear panels in U.S. Pat. No. 5,151,092, entitled “Absorbent Article With Dynamic Elastic Waist Feature Having A Predisposed Resilient Flexural Hinge”, issued to Buell et al., on Sep. 29, 1992, which is incorporated herein by reference. Accordingly, the backsheet is preferably elongatable, more preferably drawable, but not necessarily elastomeric, so that the backsheet 26 will, upon mechanical stretching, be at least to a degree permanently elongated such that it will not fully return to its original undistorted configuration. In some embodiments, the backsheet 26 can be subjected to mechanical stretching without undue rupturing or tearing. Thus, the backsheet 26 may have an ultimate elongation to break of at least 200% to about 700% in the cross-machine direction as measured using a method consistent with ASTM (American Society of Testing Materials) D-638. Films suitable for use as the backsheet 26 generally contain a high content of linear low density polyethylene. The Clopay Corporation of Cincinnati, Ohio, manufactures a suitable backsheet under the designation 1401. Other suitable materials for the backsheet 26 include RR8220 blown films and RR5475 cast films as manufactured by Tredegar Industries, Inc. of Terre Haute, Ind.

Alternatively, the backsheet 26, or any portion thereof, may comprise a structural elastic-like film (SELF) web. A structural elastic-like film web is an extensible material that exhibits an elastic-like behavior in the direction of elongation without the use of added elastic materials. The SELF web includes a strainable network having at least two contiguous, distinct, and dissimilar regions. One of the regions is configured so that it will exhibit resistive forces in response to an applied axial elongation in a direction parallel to the predetermined axis before a substantial portion of the other region develops significant resistive forces to the applied elongation. At least one of the regions has a surface-pathlength which is greater than that of the other region as measured substantially parallel to the predetermined axis while the material is in an untensioned condition. The region exhibiting the longer surface-pathlength includes one or more deformations which extend beyond the plane of the other region. The SELF web exhibits at least two significantly different stages of controlled resistive force to elongation along at least one predetermined axis when subjected to an applied elongation in a direction parallel to the predetermined axis. The SELF web exhibits first resistive forces to the applied elongation until the elongation of the web is sufficient to cause a substantial portion of the region having the longer surface-pathlength to enter the plane of applied elongation, whereupon the SELF web exhibits second resistive forces to further elongation. The total resistive forces to elongation are higher than the first resistive forces to elongation provided by the first region.

The strainable web material may be comprised of substantially of linear low density polyethylene (LLDPE). The strainable web material may also be comprised of other polyolefins such as polyethylenes, including low density polyethylene (LDPE), ultra low density polyethylene (ULDPE), high density polyethylene (HDPE), or polypropylene and blends thereof with the above and other materials. Examples of other suitable polymeric materials which may also be used include, but are not limited to polyester, polyurethanes, compostable or biodegradable polymers, heat shrink polymers, thermoplastic elastomers, and breathable polymeric structures. SELF webs suitable for the present disclosure are more completely described in the commonly assigned European Patent Application WO/1995/003765, entitled “Web materials Exhibiting Elastic-Like Behavior”, published Feb. 9, 1995 in the names of Chappel et al., which is incorporated herein by reference.

The size of the backsheet 26 may be generally dictated by the size of the absorbent core 28 and the exact diaper design selected. In one embodiment, the backsheet 26 has a modified hourglass shape extending beyond the absorbent core a minimum distance of about 1 cm to about 5 cm (about 0.5 inch to about 2 inch) around the entire periphery of the containment assembly 22.

The topsheet 24 is positioned adjacent the body-facing surface 56 of the absorbent core 28 and may be joined thereto and to the backsheet 26 by attachment means such as those well known in the art. Suitable attachment means are described with respect to joining the backsheet 26 to the absorbent core 28. In one embodiment, the topsheet 24 and the backsheet 26 are joined directly to each other in the diaper periphery. The topsheet 24 is preferably compliant, soft feeling, and non-irritating to the wearer's skin. Further, the topsheet 24 is preferably liquid pervious permitting liquids (e.g., urine) to readily penetrate through its thickness. A suitable topsheet 24 may be manufactured from a wide range of materials, such as, for example, porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. The topsheet 24 may be made of a hydrophobic material to isolate the wearer's skin from liquids which have passed through the topsheet 24 and are contained in the absorbent core 28 (i.e., to prevent rewet). If the topsheet 24 is made of a hydrophobic material, at least the upper surface of the topsheet 24 is treated to be hydrophilic so that liquids will transfer through the topsheet more rapidly. This configuration diminishes the likelihood that body exudates will flow off the topsheet 24 rather than being drawn through the topsheet 24 and being absorbed by the absorbent core 28. The topsheet 24 can be rendered hydrophilic by treating it with a surfactant. Suitable methods for treating the topsheet 24 with a surfactant include spraying the topsheet 24 material with the surfactant and immersing the material into the surfactant. A more detailed discussion of such a treatment and hydrophilicity is contained in U.S. Pat. No. 4,988,344, entitled “Absorbent Articles with Multiple Layer Absorbent Layers” issued to Reising, et al. on Jan. 29, 1991 and U.S. Pat. No. 4,988,345, entitled “Absorbent Articles with Rapid Acquiring Absorbent Cores” issued to Reising on Jan. 29, 1991, each of which is incorporated by reference herein.

In one embodiment, at least a portion of the topsheet 24 is subjected to mechanical stretching in order to provide a “zero strain” stretch laminate that forms the waist features 34. The topsheet 24 can be prestrained by any methods as are known in the art including, but not limited to, the methods described herein with respect to the formation of the “zero strain” stretch backsheet. Thus, the topsheet 24 is preferably elongatable, more preferably drawable, but not necessarily elastomeric, so that the topsheet 24 will, upon mechanical stretching, be at least to a degree permanently elongated such that it will not fully return to its original configuration. In various embodiments, the topsheet 24 can be subjected to mechanical stretching without undue rupture or tearing. Thus, the topsheet 24 may have a low cross-machine direction (lateral direction) yield strength.

There are a number of manufacturing techniques which may be used to manufacture the topsheet 24. For example, the topsheet 24 may be a nonwoven web of fibers. When the topsheet 24 comprises a nonwoven web, the web may be spunbonded, carded, wet-laid, melt-blown, hydroentangled, combinations of the above, or the like. A suitable topsheet 24 is carded and thermally bonded by means well known to those skilled in the art. A satisfactory topsheet 24 comprises staple length polypropylene fibers having a denier of about 2.2 As used herein, the term “staple length fibers” refers to those fibers having a length of at least about 15.9 mm (0.625 inches). In one embodiment, the topsheet 24 has a basis weight from about 14 to about 25 grams per square meter. A suitable topsheet is manufactured by Veratec, Inc., a Division of International Paper Company, of Walpole, Mass. under the designation P-8.

The diaper 20 may further comprise elasticized leg cuffs 32 for providing improved containment of liquids and other body exudates. Each elasticized leg cuff 32 may comprise several different embodiments for reducing the leakage of body exudates in the leg regions. The leg cuffs can be and are sometimes also referred to by other names, such as leg bands, side flaps, barrier cuffs, or elastic cuffs, for example. U.S. Pat. No. 3,860,003 describes a disposable diaper which provides a contractible leg opening having a side flap and one or more elastic members to provide an elasticized leg cuff (gasketing cuff). U.S. Pat. No. 4,909,803, entitled “Disposable Absorbent Article Having Elasticized Flaps” issued to Aziz et al. on Mar. 20, 1990, describes a disposable diaper having “stand-up” elasticized flaps (barrier cuffs) to improve the containment of the leg regions. U.S. Pat. No. 4,695,278, entitled “Absorbent Article Having Dual Cuffs” issued to Lawson on Sep. 22, 1987, describes a disposable diaper having dual cuffs including a gasketing cuff and a barrier cuff. U.S. Pat. No. 4,704,115, entitled “Disposable Waste Containment Garment” issued to Buell on Nov. 3, 1987, discloses a disposable diaper or incontinence garment having side-edge-leakage-guard gutters configured to contain free liquids within the garment. Each of these patents is incorporated herein by reference. While each elasticized leg cuff 32 may be configured so as to be similar to any of the leg bands, side flaps, barrier cuffs, or elastic cuffs described above, each elasticized leg cuff 32 may comprise a gasketing cuff as described in the above-referenced U.S. Pat. No. 3,860,003 and a barrier cuff as described in the above-referenced U.S. Pat. No. 4,909,803.

The diaper 20 may further comprise a waist feature 34 that helps provide improved fit and containment. The waist feature 34 may be elastically extensible or inelastic. In some embodiments, the waist feature 34 has at least some ability to expand and contract in conjunction with wearer's motions. The waist feature 34 may extend longitudinally outwardly from at least one of the waist edges 60 of the absorbent core 28 and generally forms at least a portion of the end edge 52 of the diaper 20. Disposable diapers are generally constructed so as to have two waist features, one positioned in the rear waist region 44 and one positioned in the front waist region 46, although diapers can be constructed with a single waist feature. Further, while the waist feature 34 or any of its constituent elements can comprise a separate element affixed to the diaper 20, the waist feature 34 may be constructed as an extension of one or more of the other elements of the diaper such as one or more layers of the backsheet 26 or the topsheet 24 or combinations thereof, for example.

The waist feature 34 may comprise any material suitable for application in a garment to be fitted to a wearer. If the waist feature 34 is to be elastic, any suitable stretchable or elastomeric materials may be used. Suitable elastomeric materials for use as the waist feature 34 are described herein below with respect to the side panels 62 and 38. The waist feature 34 may be constructed in a number of different configurations including those described in U.S. Pat. No. 4,515,595, entitled “Disposable Diapers with Elastically Contractible Waistbands” issued to Kievit et al. on May 7, 1985 and the above referenced U.S. Pat. No. 5,151,092 issued to Buell on Sep. 29, 1992; each of these references being incorporated herein by reference. Further, the waist feature 34 may comprise “pleats” that may be activated by the diaperer to provide additional extension in the waist regions. (As used herein, the term “pleats” refers to regions of material that have been folded back upon themselves such that when an activation force is applied the material unfolds.)

Referring to FIGS. 1-5, one of the front or rear waist regions 46 or 44 comprises two side panels, such as the first side panel 62a, and the second side panel 62b, which typically form the outer lateral portions of one of the front or rear waist regions 44 or 46. A four-panel execution is illustrated in FIG. 1, with the first side panel 62a and second side panel 62b positioned in a first waist region, for example the rear waist region 44. The longitudinally opposing waist region may also include a pair of laterally opposing side panels, such as third and fourth panels 38a, 38b, which may form the outer lateral portions of the opposing waist region, for example, the front waist region 46. While a four-panel execution is shown in FIG. 1, in various embodiments, only one waist region may comprise lateral portions that form side panels. For example, some embodiments may only have third and fourth panels 38a, 38b in the front waist region 46 and the rear waist region 44 will not have side panels. Alternatively the absorbent article may only have first and second side panels 62a, 62b in the rear waist region 44 and the front waist region 46 will not have side panels. Any description herein referring to qualities, characteristics, and arrangements of the first and second side panels 62a, 62b is also applicable to the third and fourth panels 38a, 38b for embodiments that utilize such panels.

As is to be appreciated, and as discussed in more detail below, the diaper 20 may comprise one or more fastening members positioned on one or more of the first and second panels 62a, 62b, the third and fourth panels 38a, 38b, or other components of the diaper 20, such as the containment assembly 22. For example, first and second closure members 30, 31 may be disposed on or form a portion of first and second side panels 62a and 62b or third and fourth side panels, 38a, 38b, in a first waist region and the third closure member, or mating closure member, may be disposed on or form a portion of the containment area 22 in the longitudinally opposing waist region. All such variations of the arrangements of the fastening system 200 are intended to be included within the scope of the disclosure.

The first closure member 30 may comprise an engaging component 202 and may be disposed adjacent the distal edge 68 on the inner surface 67 of a side panel. The closure members 30 and 31 may either be a discrete separate element affixed to the diaper 20 or a unitary piece of material that is neither divided nor discontinuous with an element of the diaper 20, such as, for example, the side panels, the backsheet, topsheet or leg cuffs. In one embodiment, when the diaper 20 is constructed, the closure members 30 and 31 engages the third closure member 33 to fasten the rear waist region 44 to the front waist region 46. The closure members 30 and 31 may comprise one or more separate fastening elements. The closure member 33 is intended to engage with the closure members 30 and 31 and closure member 33 may comprise one or more separate fastening elements. In addition, any of the closure members may be disposed on or form a portion of one of the surfaces of the diaper 20.

The third closure member 33 may comprise one or more landing components 204 disposed in the waist region longitudinally opposing the waist region comprising the first and/or second closure members. In some embodiments, the third closure member 33 may comprise one or more engaging components. The third closure member 33 may be disposed on or form a portion of the garment facing surface 40 of the diaper 20. The third closure 33 member may be unitary with other elements of the diaper 20, such as the topsheet 24, the backsheet 26, the leg cuffs, the various side panels or combinations thereof. In one alternative embodiment, the third closure member 33 comprises at least two separate discrete patches or areas in the front waist region 46. Various configurations of the closure members 30, 31 and 33 can help to ensure a proper waist circumference and proper tensioning for the diaper to perform as either a conventional diaper or a pull-on. In one embodiment an absorbent article providing proper tensioning and fit can be provided by the use of a single third closure member 33 with indicia indicating the proper positioning of each of closure members 30 and 31 thereon. (Discrete individual landing components 204, as shown in FIGS. 1-5, may also be provided with indicia thereon.)

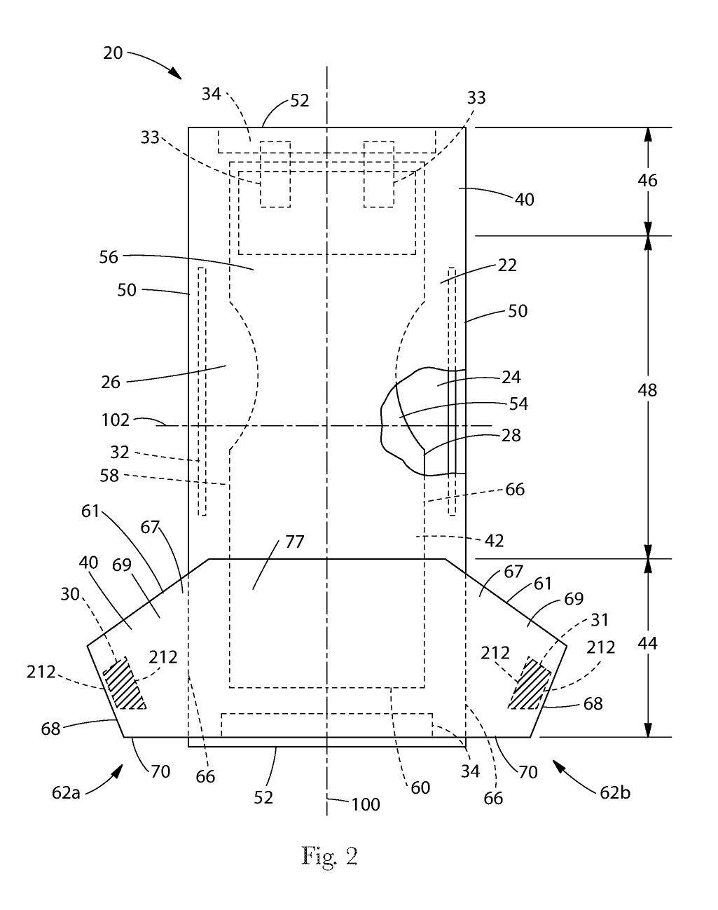

The third closure member(s) 33 may take on any suitable shape or size. In one embodiment, as shown in FIG. 2, the third closure 33 member comprises two distinct landing components which are generally in the shape of parallelograms. In such embodiments, it may be desirable for the landing components 204 comprised in the third closure member 33 to be configured such that their major axis is at an angle to both the longitudinal centerline 100 and the lateral centerline 102 of the absorbent article. In one embodiment, the major axis of the landing components 204 are angled laterally outwardly as it extends away from the lateral centerline 102 towards the end edge 52 in the front waist region 46. The landing components 204 of the third closure member 33 may also be configured so as to correspond to the angle of the first and second closure members 30 and 31 when the diaper is fastened about the wearer. This ensures that a sufficient portion of each closure member engages with each corresponding landing component. (Embodiments are contemplated, however, wherein the first and second closure members 30 and 31 are not necessarily configured such that they correspond to the angle of the landing component(s) 204. In such cases, the diaperer is left with the flexibility to fasten the fastening system 200 such that the engaging components 202 completely overlap the landing component(s) 204 or not.)

FIG. 2 illustrates an embodiment of the diaper 20 comprising first and second side panels 62a, 62b extending laterally outward from the side edges 50 of the containment assembly 22 in rear waist region 44. This embodiment of the diaper 20 may comprise substantially similar components to the four-panel execution of the diaper 20. Referring to FIGS. 1-3, the first and second side panels 62a, 62b may provide structural elements that the user may hold while fastening or refastening the diaper 20 about the wearer in a conventional taped diaper configuration. As is to be appreciated, if third and fourth panels 38a, 38b are used, these panels may provide structure elements that the user may hold while fastening or refastening the diaper 20 about the wearer in a conventional taped diaper configuration.

The first and second side panels 62a, 62b may be disposed at or adjacent one of the longitudinal extending edges 50 of the containment assembly 22, such as in at least a portion of the rear waist region 44. While it is not necessary that the laterally opposed pair of side panels be identical, as shown in FIG. 1, in one embodiment they are mirror images of one another. The first and second side panels 62a, 62b may comprise portions of other elements of the absorbent article or may be separate elements joined with the containment assembly 22. Each of the first and second side panels 62a, 62b, depending on their shape, may have a longitudinally proximal edge 61, a longitudinally distal edge 70, a laterally distal edge 68, an inner surface 67, and an outer surface 69.

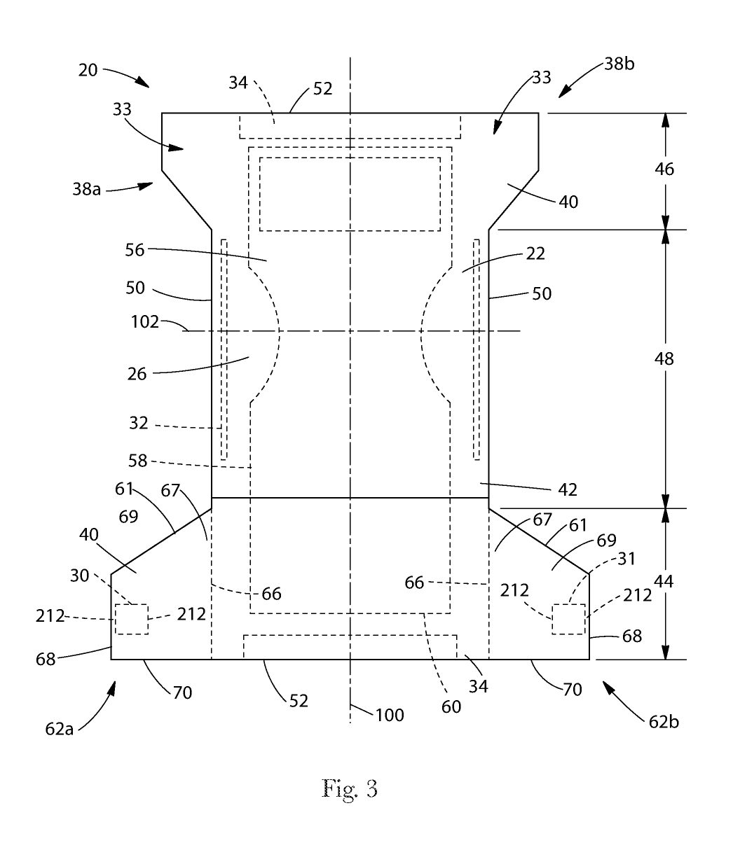

In some embodiments, as illustrated in FIG. 2-3, the first and second side panels 62a, 62b may be attached or joined to the containment assembly 22. In such embodiments, the first and second side panels 62a, 62b may have a laterally proximal edge 66, in addition to the longitudinally proximal edge 61, the longitudinally distal edge 70, and the laterally distal edge 68. The laterally proximal edge 66 of the side panel may be disposed at/or adjacent one of the side edges 50 of the containment assembly 22 and the laterally distal edge 68 spaced laterally outwardly from the proximal edge 66. As illustrated in FIG. 2, the side panels 62a, 62b may be extensions of a belt-like element 77. The belt-like element 77 may extend across the containment assembly 22 of the diaper 20 and laterally outwardly therefrom to form the side panels 62a, 62b. The belt-like element 77 may be integral with other elements of the diaper or may be a separate element attached thereto. Additionally, as illustrated in FIG. 3, the side panels 62a, 62b may be joined to the containment assembly 22. The attachment region 73 may be generally long the side edges 50. As is to be appreciated the third and fourth panels 38a, 38b may also be provided using an element similar to the belt-like element 77 or attached to the containment assembly 22 along the side edges 50. If the belt-like element 77 or the side panels 62a, 62b are separate elements joined to the diaper 20, they can be joined by any by any means as known in the art. Examples of suitable attachment means include adhesive bonding, heat bonding, pressure bonding, ultrasonic bonding, dynamic mechanical bonding or a combination of any of these means or any other means as are known in the art.

In one embodiment, the longitudinally proximal edges 61 of the first and second side panels 62a, 62b form portions of the leg openings of the diaper 20 while the longitudinally distal edges 70 of the first and second side panel 62a, 62b forms a portion of the waist opening of the diaper 20. As shown in FIGS. 1-3, the outer surface 69 of each side panel is that surface which faces away from the wearer when the diaper is being worn and generally corresponds to the garment facing surface 40 of the containment assembly 22. As shown in FIGS. 1-3, the inner surface 67 of each side panel is that surface which faces the wearer when the diaper 20 is being worn and generally corresponds to the body facing surface 42 of the containment assembly.

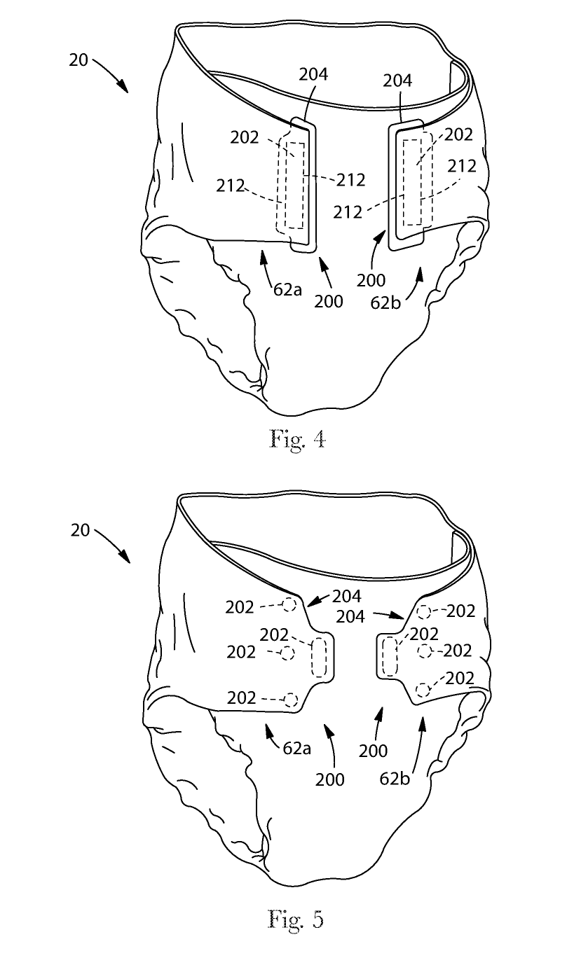

Referring to FIGS. 4-5, the diaper 20 may be fitted to the wearer in a pull-on (or pant) configuration. The diaper may be constructed generally as stated above before the diaper is placed on the wearer. Accordingly, in such a configuration, the engaging components 202 may be engaged with the landing component 204 to form a pant-like article having a waist hoop and a pair of leg openings. In this configuration, the wearer's legs are directed through the leg openings. Once the wearer's legs are through the leg openings, the diaperer can then pull the waist hoop of the diaper up over the wearer's hips such that the waist hoop encircles the waist of the wearer. (It should be noted that the wearer may be the diaperer in some cases, especially when the wearer is a child in the toilet training stage or when the wearer is an adult.)

The diaper 20 may alternatively be applied to the wearer in a conventional (or taped diaper) configuration. To do so, the rear waist region 44 is preferably placed under the wearer's back. The remainder of the diaper 20 is preferably then drawn between the wearer's legs such that the other waist region, preferably the front waist region 46, is positioned across the front of the wearer. The diaperer preferably then grasps the first side panel 62a or the second side panel 62b, or both and wraps them around the waist of the wearer. In one embodiment, the diaperer then engages the engaging components 202 disposed in the back waist region with the landing component 204 disposed on in the front waist region 46. This forms a waist closure on each side of the wearer and completes the construction of the diaper in a conventional configuration.

Of course, the diaper 20 may be fitted to the wearer in any other suitable manner, including a combination of the steps described above with regard to the conventional and pull-on embodiments. When the diaper 20 is used as a pull-on, the side panels are prefastened as described above to form a pant-like article, as shown in FIGS. 4-5. In one embodiment, the side panels are prefastened as packaged and prior to being handled by the diaperer. In any configuration, the refastenable fastening system 200 provides the user with easy access to inspect the diaper 20 for soiling. Further, the fastening system 200 gives the user the option as to how the diaper 20 will be removed. The diaper 20 can be removed by pulling the diaper 20 down and off the wearer without disengaging the elements of the fastening system 200, or by disengaging at least one of the engaging components 202 form its corresponding, i.e. mating, landing component 204 and removing the diaper 20 from around the wearer's waist. Thus, the fastening system 200 maintains the diaper in the pull-on configuration and permits the user to fit the diaper 20 to the wearer in at least two different configurations, a conventional taped diaper configuration and a pull-on configuration. In either configuration, the diaper 20 can be removed by pulling the diaper down in its fully constructed configuration or by opening the closures formed by the fastening system 200.

The fastening system 200 may comprise any attachment means known in the art including pressure sensitive adhesives, cohesive materials, mechanical fasteners such as hook and loop type fasteners, or any combination of these or any other attachment means known in the art. As used herein, the term “hook and loop type fasteners” refers to fastening means comprising an engaging component and a complementary landing component. The term “hook” is used to designate a material having engaging elements. Thus, the hook fastening material may also be referred to as a male fastener. It should also be understood that the use of the term “hook” should be non-limiting in the sense that the engaging elements may comprise any shapes as are known in the art so long as they are adapted to engage a complementary landing component. In some embodiments, nonwoven materials forming all or a part of a surface of any of the side panels may function as a fastening component, such as a landing component, much like a loop material in a hook and loop mechanical fastener. Exemplary adhesive tape tab fastening systems are disclosed in U.S. Pat. No. 3,848,594, entitled “Tape Fastening System for Disposable Diaper” issued to Buell on Nov. 19, 1974; and U.S. Pat. No. 4,662,875, entitled “Absorbent Article” issued to Hirotsu and Robertson on May 5, 1987. Exemplary fastening systems comprising mechanical fastening components are described in U.S. Pat. No. 5,058,247, entitled “Mechanical Fastening Prong” issued to Thomas Oct. 22, 1991; U.S. Pat. No. 4,869,724, entitled “Mechanical Fastening Systems With Adhesive Tape Disposal Means For Disposal of Absorbent Articles” issued to Scripps on Sep. 26, 1989; and U.S. Pat. No. 4,846,815, entitled “Disposable Diaper Having an Improved Fastening Device” issued to Scripps on Jul. 11, 1989. An example of a fastening system having combination mechanical/adhesive fasteners is described in U.S. Pat. No. 4,946,527, entitled “Pressure-Sensitive Adhesive Fastener and Method of Making Same” issued to Battrell on Aug. 7, 1990. Each of these patents is incorporated herein by reference.

In various embodiments, the fastening system 200 may comprise an engaging component 202 and a complementary landing component 204. The landing component 204 may be manufactured from a wide range of materials and configurations capable of securely engaging the engaging component 202. For example, the landing component 204 may comprise identical complementary elements or distinct complementary elements. As used herein, the term “identical complementary elements” is used to define mechanical fastening systems wherein the engaging elements of the engaging component 202 and the landing component 204 comprise the same configuration or structure. Examples of such systems are described in Brown et al. U.S. Pat. No. 4,322,875, entitled “Two Strip Materials Used For Forming Fasteners” issued on Apr. 16, 1982 and Kellenberger et al. U.S. Pat. No. 4,701,179, entitled “Fixed Position Fasteners For Disposable Absorbent Garments” issued on Oct. 20, 1987. The term “distinct complementary elements” is used herein to designate a system wherein the engaging component 202 is different from the landing component 204 but is engageable therewith.

In various embodiments, the longitudinal length of the engaging component 202 is substantially less than the longitudinal length of the side panel to which it is attached or otherwise disposed. The longitudinal length of the side panel is measured generally parallel to the longitudinal axis from the longitudinally distal point of the fastener to the longitudinally proximal point of the fastener. If a continuous engaging component 202 is utilized, the longitudinal length of the engaging component may be measured generally along the longitudinal extending edge 212. If a plurality of discrete engaging components 202 are utilized on a single side panel, the longitudinal length of the engaging component is the summation of the longitudinal length of each discrete engaging component 202. In one embodiment, the longitudinal length of the engaging component 202 may be less than about 80% of the longitudinal length of an associated side panel. In one embodiment, the longitudinal length of the engaging component 202 may be less than about 70% of the longitudinal length of an associated side panel. In one embodiment, the longitudinal length of the engaging component 202 may be less than about 50% of the longitudinal length of an associated side panel.

Referring to FIGS. 1-5, an engaging component 202 including a hook fastening material may have a base 208 and a plurality of engaging elements 206 extending from the base 208. The hook fastening material is intended to engage fibrous elements of a loop fastening material so as to provide a secure fastening device. Thus, the hook fastening material may be manufactured from a wide range of materials. Further, the engaging elements 206 may have any shape such as hooks, “T's”, “mushrooms” or any other shape as are well known in the art. Suitable materials include nylon, polyester, polypropylene, or any combination of these materials. Examples of hook fastening materials are available from Aplix Inc. of Charlotte, N.C. under the trade designation 960, 957 and 942. Other hook fastening materials are available from the Minnesota Mining and Manufacturing Company of St. Paul, Minn. under the trade designations CS200, CS300, MC5 and MC6. Yet another hook fastening material is described in U.S. Pat. No. 5,058,247, entitled “Mechanical Fastening Prong” issued to Thomas Oct. 22, 1991, which is incorporated herein by reference.

The engaging components 202 may be joined to the absorbent article by any suitable means. They may be directly joined with any portion of the side panels 62 and/or side panels 38, such as the material comprising the inner surface 67 of the side panel, or the material comprising the outer surface 69 of the side panel, or mounted on some intermediate member, such as a tape tab or other tab member. The engaging components may also be joined to the interior or exterior surface of the containment assembly 22. The engaging component, tab, intermediate member or combination thereof may be joined to a side panel or absorbent assembly by any means known in the art, including, but not limited to heat, pressure, ultrasound, adhesive, cohesive or any combination of these or other bonding means.

The landing component 204 may comprise a plurality of fiber elements, such as a loop fastening material, that engage the engaging elements 206 of the engaging component 202. The loop fastening material may be manufactured from a wide range of materials to provide fiber elements, such as loops. Suitable materials include woven materials, nonwovens, nylons, polyesters, polypropylenes, or any other materials comprising a plurality of fiber elements or combination of these materials. Such nonwoven materials would include the nonwoven materials used to form a portion of the side panel as well as nonwovens disposed on the backsheet 26. One suitable loop fastening material is a nonwoven available from Minnesota Mining and Manufacturing Company, St. Paul, Minn. under the trade designation EBL. One loop fastening material comprises a number of shaped engaging elements projecting from a woven backing such as the commercially available material designated Guilford 18904 available from Guilford Mills of Greensboro, N.C. Other suitable landing components 204 are available from the Minnesota Mining and Manufacturing Company of St. Paul, Minn. under the trade designation KLT. An inexpensive loop fastening material and a method of making the same is described in U.S. Pat. No. 5,032,122, entitled “Loop Fastening Material For Fastening Device and Method of Making Same” issued to Noel et al., Jul. 16, 1991, which is hereby incorporated herein by reference. Another suitable landing component material is described in U.S. Pat. No. 5,326,612, entitled “Nonwoven Female Component for Refastenable Fastening Device and Method of Making the Same” issued to David J. K. Goulait on Jul. 5, 1994, which is hereby incorporated by reference herein. Yet other suitable landing components are described in U.S. patent application Ser. No. 08/254,814, entitled “Nonwoven Female Component For Refastenable Fastening Device and Method of Making the Same” filed Jun. 6, 1994 in the names of Patrick Jay Allen et al.; U.S. patent Ser. No. 08/287,571, entitled “Nonwoven Female Component For Refastenable Fastening Device” filed Aug. 9, 1994 in the names of Willie F. King et al.; and U.S. patent Ser. No. 08/374,269, entitled “Female Component For Refastenable Fastening Device” filed Jan. 18, 1995 in the names of Carl L. Bergman et al. All of these patent applications are incorporated herein by reference.

The landing component 204 may be joined with the diaper 20 by any means known in the art, including but not limited to adhesives, cohesives, heat, pressure, ultrasound, or any combination thereof. Further, the specific bonding patterns described above with regard to the engaging components 202 may be used to join the landing component(s) 204 to the diaper 20. The landing component(s) 204 may be joined with the diaper 20 by means of an adhesive bond which may cover any portion of its backing.

Still referring to FIGS. 1-5, the elements of the fastening system 200 may be disposed on either the garment facing surface 40 or the body facing surface 42 of the diaper 20 or any of the elements forming the diaper, such as, for example any of the side panels 62a, 62b, 38a, 38b or the containment assembly 22. In one embodiment the engaging component 202 is disposed on the outer surface 69 (i.e., garment facing surface) of the side panels in one waist region and the landing component is disposed on or forms a portion of the inner surface 67 (i.e., wearer facing surface) of the side panels in the opposing waist region. Thus, in one embodiment, the engaging component 202 may be disposed on the outer surface 69 of the third and fourth panels 38a, 38b and the landing component 204 is disposed on the inner surface 67 of the first and second panels 62a, 62b. In an alternative embodiment, the engaging component 202 is disposed on the inner surface 67 (i.e., wearer facing surface) of the side panels in one waist region and the landing component 204 is disposed on or forms a portion of the outer surface 69 (i.e., garment facing surface) of the side panels in the opposing waist region. In yet another alternative embodiment, the engaging component 202 is disposed on the inner surface 67 (i.e., wearer facing surface) of the side panels in one waist region and the landing component 204 is disposed on or forms a portion of the outer surface 69 (i.e., garment facing surface) of the containment assembly 22 in the opposing waist region. In still another alternative embodiment, the engaging component 202 is disposed on the garment facing surface of the containment assembly 22 in one waist region and the landing component 204 is disposed on or forms the inner surface 67 (i.e., wearer facing surface) of the side panel in the opposing waist region. In one embodiment, the fastening elements are disposed on the diaper 20 such that the fasteners do not irritate the wearer's skin. It should be noted that the engaging components 202 and the landing component(s) 204 may comprise one or more individual fastening elements and may be of any shape, pattern and size. Further, embodiments are contemplated wherein the engaging components 202, the landing components 204, or both are not continuous, but rather include a number of individual members that provide the requisite fastening characteristics and to allow for the best fit for a broad range of wearers.

In some embodiments, the longitudinal edges 212 of the engaging components 202 may be generally parallel with the longitudinal centerline 100 of the absorbent article, as shown in FIGS. 1 and 3, or may be at an angle to the longitudinal centerline 100, as shown in FIG. 2, for example. An alternative to angling the engaging components 202 is to fasten the engaging components 202 to their corresponding landing components 204 at an angle. Providing the engaging components 202 at an angle to the longitudinal centerline 100 of the absorbent article provides at least two potential benefits. When the engaging components 202 are angled, as shown in FIG. 2, the engaging components 202 are located such that skin marking on the upper thigh is generally avoided, despite the motions of the wearer. Also, with this approach, the entire fastener can fit into and not straddle the wearer's leg crease.

The side panels 38, 62 may take on a number of different sizes, shapes, configurations, and materials. The side panels 38, 62 may be rectangular or trapezoidal in shape, for example. The side panels 38, 62 may have one or more straight edges and one or more curved edges to provide a proper appearance and fit relative to the wearer's body. The side panels 38 and/or the side panels 62 may comprise a portion of the material making up one or more of the diaper elements, such as the elastic leg cuff, the topsheet and/or one or more layers of the backsheet, and combinations thereof. Alternatively, the side panels 38 and/or the side panels 62 may be formed by a separate element or a plurality of elements affixed to the diaper, i.e. the containment assembly.

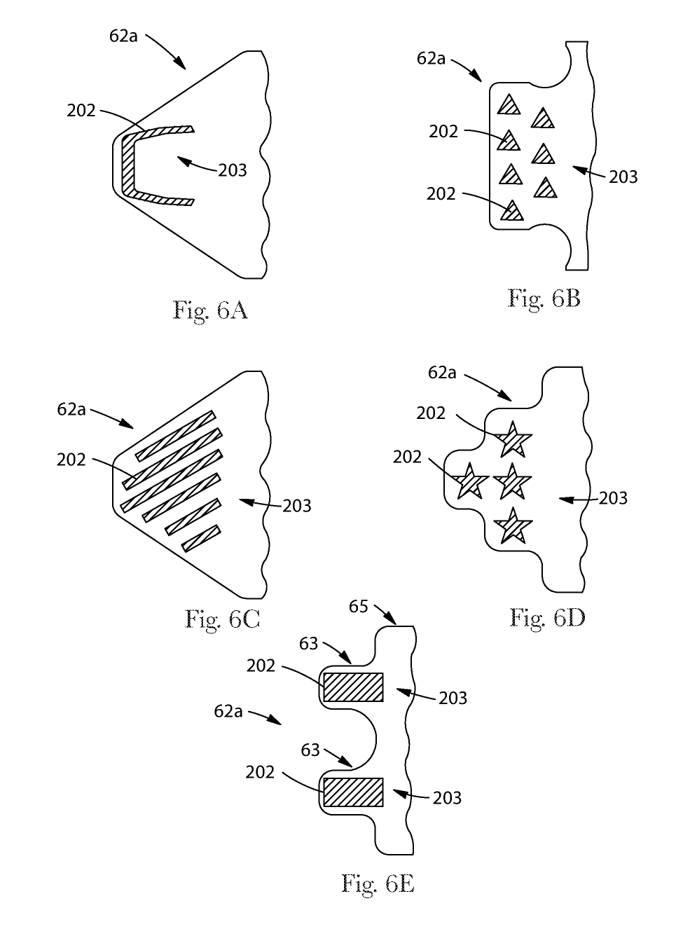

FIGS. 6A-6E illustrate side panel configuration in accordance with various non-limiting embodiments. The first side panel 62a with the engaging component 202 is illustrated as a representative side panel of the diaper 20. It is to be appreciated that any description herein referring to the first side panels 62a is also applicable to the second side panel 62b, and the third and fourth panels 38a, 38b in embodiments that utilize such panels. As illustrated, the first side panel 62a may be any suitable shape. As shown in FIG. 6E, for example, the side panel 62a may have one or more extensions 63, such as tabs or protrusions, generally extending from a body portion 65. The first side panel 62a may provide an engaging component 202, a landing component 204, or a combination thereof. FIGS. 6A-6E illustrate only engaging components 202. The engaging component 202 may be arranged in any suitable configuration on the first side panel 62a to form a zone of fastening 203. The zone of fastening 230 is generally the area of the side panel surrounding the engaging component(s) 202. As illustrated, the engaging component 202 may be discontinuous in at least one of the longitudinal and lateral direction. Furthermore, the size, shape, and/or area of the engaging component 202 may be configured to provide adequate force distribution along the side panels 62a, 62b, 38a and 38b in the longitudinal direction when subjected to the various forces encountered with the diaper 20 is worn by a wearer. In various embodiments, the engaging component 202 has a longitudinal length that is substantially less than the longitudinal length of the side panel that provides the engaging component 202. In some embodiments, the shape and/or closure arrangement of the various side panels of the diaper 20 may differ. For example, in one embodiment the first and second side panels 62a, 62b have a different shape than the third and fourth panels 38a, 38b. In another embodiment the first and second side panels 62a, 62b have a similar shape to the third and fourth panels 38a, 38b. The side panels 38, 62 may take on a number of different sizes, shapes, configurations and materials. Thus, the exact length, width and thickness of the side panels 38, 62 may vary depending on the intended user. In various embodiments, the side panels 38 and 62 may be substantially rectangular in shape. The side panels 38 and 62 may have the same lateral extent as measured from the side edge 50 of the containment assembly 22 to the laterally distal edge 68 of the side panel. The side panels may also have the same longitudinal extent as measured from the proximal longitudinal edge 61 to the distal longitudinal edge 70.

In one embodiment the side panels may have a longitudinally distal edge 70 that is substantially linear and a longitudinally proximal edge 61 that is substantially linear and non-parallel to the longitudinally distal edge 70. In an alternative embodiment, the side panels may have a longitudinally distal edge 70 that is substantially linear and a longitudinally proximal edge 61 that is substantially arcuate. In yet another alternative embodiment, the side panel may have a laterally proximal edge 66 that is substantially linear and a laterally distal edge 68 that is substantially linear and non-parallel to the laterally proximal edge 66. In one embodiment, the side panel 62a, 62b may be separate elements joined to the diaper 20, they can be joined by any by any means as known in the art. Examples of suitable attachment means include adhesive bonding, cohesive bonding, heat bonding, pressure bonding, ultrasonic bonding, dynamic mechanical bonding or a combination of any of these means or any other means as are known in the art.

The side panels may also comprise extensible or non-extensible material. In one embodiment, the side panels comprise multi-directional extensible side panels. Such side panels are described in detail in copending U.S. patent application Ser. No. 08/155,048, entitled “Absorbent Article With Multi-Directional Extensible Side Panels”, filed on Nov. 19, 1993 in the names of Miguel Robles et al., which is hereby incorporated by reference herein. Suitable materials for use in the side panels include woven webs; nonwoven webs; films including polymeric films; elastomeric films, foams, laminate materials including film laminates, nonwoven laminates, or zero strain laminates; formed films; elastomers; composites; elastic strands, structural elastic like-film (SELF) webs or any combination of materials hereinafter described. The side panels may be joined to the containment assembly 22 by any means as known in the art; for example the side panels may be continuously or intermittently bonded to the containment assembly 22 using adhesive, heat bonding, pressure bonding, ultrasonic bonding, dynamic mechanical bonding or any other method that is known in the art.

It has been found that in order for the diaper 20 to work effectively as a pull-on or as a conventional diaper capable of fitting a wide range of wearers that the first and second side panels 62a, 62b and/or the third and fourth side panels 38a, 38b, in conjunction with the waist feature(s), 34 should provide sustaining forces within a definite range at certain waist hoop circumferences. (As used herein, the term “sustaining forces” refers to the inwardly directed forces that hold the diaper against the waist and hips of the wearer so as to reduce sagging or gapping during use. The sustaining force will often be less than the force needed to stretch the material to the length in which the sustaining force is measured. The term “waist hoop circumference” and “hip hoop circumference” refers to the circumference of the waist of the diaper when it is fully constructed into a pant-like article, either before being pulled onto the wearer or after being fitted to the wearer in the conventional configuration.) In some embodiments, the waist hoop circumference of the diaper 20 in a relaxed state (i.e. having no tension applied) is smaller than the waist of the smallest wearer in the size range. Likewise, the waist hoop circumference of the diaper 20 in a fully stretched condition may be larger than the circumference of the waist of the largest expected wearer in the particular size range. (As used herein, the term “fully stretched” means that the waist hoop of the diaper has been stretched to its elastic limit, at which point any further force applied would deform, destroy or otherwise alter the structure of at least a portion of the material. Alternatively, the term “fully stretched” may refer to the waist of the diaper when the side panels have been stretched to a degree that any further stretching would be unacceptable for such a product, generally greater than 3500 grams.) This ensures that the diaper will properly fit all wearers in any size range and will function properly as a pull-on/off article.

Alternatively, the stretch characteristics of the diapers of the present disclosure can be defined in terms of the amount of extension that the side panels, such as side panels 62 and/or side panels 38, provide while providing certain recovery forces. (As used herein, the term “recovery force” is used to denote the load which can be obtained from the recovery or relaxation curve of a stress/strain graph.) Further, the elastomeric portion of each side panel may be capable of extending between about 150% and about 300% when a elongation force of 3000 grams or less is applied. (The percentages described above are calculated by subtracting the relaxed state length from the extended length, dividing that number by the relaxed state length and multiplying the result by 100%.) In some embodiments, each side panel has an elastomeric portion having a relaxed state length which is defined as the length of the elastomeric portion of the side panel measured along an axis that is generally parallel to the transverse centerline 102 and typically corresponds to the direction of forces that are applied to the side panel during use.

In one embodiment, the first and second side panels 62a, 62b at least partially elastomeric or elastically extensible in the lateral direction to provide the necessary stretch characteristics to work effectively as a pull-on article and fit a wide range of user sizes. The terms “elastomeric” or “elastically extensible” refer to materials that extend in at least one direction when a force is applied to the material, and return to approximately their original dimensions after the force is released. The “lateral direction” is defined as the direction parallel to the transverse centerline 102 of the diaper. Furthermore, if third and fourth side panels 38a, 38b are utilized, they may be least partially elastomeric or elastically extensible in the lateral direction. In some embodiments, only one pair of side panels will be at least partially elastomeric or elastically extensible in the lateral direction. For example, in one embodiment the third and fourth side panels 38, 38b are elastically extensible, while the first and second side panels 62a, 62b are not. In another embodiment the third and fourth side panels 38, 38b are not elastically extensible, while the first and second side panels 62a, 62b are elastically extensible. In yet another embodiment, third and fourth side panels 38, 38b are elastically extensible and first and second side panels 62a, 62b are also elastically extensible. Generally, elastomeric side panels also provide more effective application of the diaper since even if the diaperer fits the diaper to the wearer asymmetrically, the diaper may self adjust during wear to attain an improved fit. Further, elastically extensible side panels provide improved dynamic fit about the waist of the wearer, reducing the possibility of sagging or gapping, and sustaining the fit of the diaper throughout the time of wear.

One elastically extensible material that has been found to be especially suitable for use in the side panels is a laminate comprising at least one cover layer joined with an elastomeric film. (As used herein, the term “cover layer” refers to any woven or nonwoven materials.) Examples of suitable cover layer materials are hereinbefore discussed with respect to the topsheet 24 and the backsheet 26. Other suitable cover layer materials include nonwovens such as Fiberweb E004203 available from Fiberweb North America of Simpsonville, S.C., and Veratec 7 pt., P-8 and P-14 available from Veratec Nonwoven Group of the International Paper Company of Walpole, Wash. Examples of suitable elastomeric films include Clopay 2870, a styrene block copolymer available from the Clopay Corporation of Cincinnati, Ohio, and Exxon 550 available from the Exxon Chemical Company of Lake Zurich, Ill. Examples of suitable synthetic foams for joining between the cover layers include: a) cross-linked natural rubber foams preferably having a caliper of approximately 50 mils and a density of approximately 13.3 pounds per cubic foot (0.214 grams per cubic cm), such as is available from Fulflex Inc., of Middletown, R.I., or as available from Ludlow Composites Corporation of Freemont, Ohio and marketed under the tradename Baby Foam; or b) polyurethane foams having a caliper of approximately 80 mils and a density of approximately 2.06 pounds per cubic foot (0.033 grams per cubic cm), such as is available from Bridgestone of Yokohama, Japan and marketed under the tradename Bridgestone SG Polyurethane Foam. Other suitable materials for use as or in the side panels include structural elastic-like film (SELF) webs, as described above, natural rubber strands, natural rubber foams, elastomeric scrims, woven or nonwoven elastomeric webs, elastomeric composites such as elastomeric nonwoven laminates, zero strain stretch laminates, prestrained stretch laminates or the like. The above referenced U.S. Pat. No. 5,151,092 issued to Buell et al., on Sep. 29, 1992, describes suitable zero strain stretch laminates and prestrained stretch laminates, and is herein incorporated by reference.

In an alternative embodiment, the side panels, such as the first and second side panels 62a, 62b and/or the third and fourth side panels 38a, 38b, may comprise a laminate including an elastomeric scrim material. In one such embodiment, the side panels may comprise a laminate of a scrim laminated between nonwovens, films, scrims, laminates or any combinations thereof. Suitable nonwovens include those mentioned above, as well as the carded polypropylene RMS 66265 available from Veratec Nonwoven Group of the International Paper Company of Walpole, Wash.; and the spunbond polyethylene available from Corovin GmbH of Germany under the trade name COROLIND. A suitable scrim is available from Conwed Plastics under the trade name REBOUND ELASTOMERIC NETTING, having between 2 and 20 strands per inch extending in the machine direction and the cross-direction. In yet another embodiment, the side panels 62a, 62b and/or the third and fourth side panels 38a, 38b may comprise a laminate including a formed film such as X-15301 manufactured by Tredegar Film Products, Inc. of Terre Haute, Ind.

One example material for the side panels has a relatively flat stress-strain curve between 50% and 200% elongation to sustain product fit on a baby, for example, and make the diaper relatively easy to apply. One material that works especially well is a laminate made with elastomeric scrim (TN2510) from Conwed Plastics. The laminate typically consists of 3-5 layers, including the scrim. The layers other than the scrim may comprise nonwovens, films or apertured films. The laminate may be bonded by any means known in the art for joining layers of a laminate. Examples of suitable bonding means include, but are not limited to, heat, pressure, ultrasound, adhesive, cohesive and coextrusion.

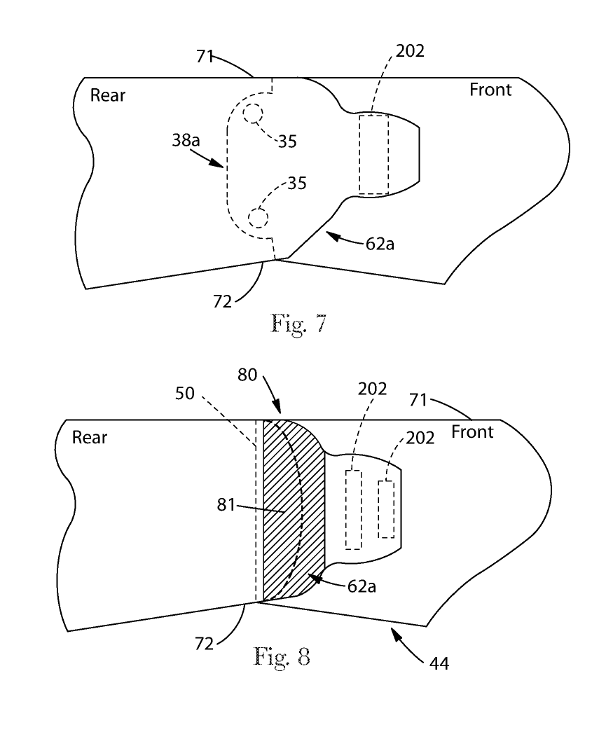

The side panels 38 and/or the side panels 62 preferably encircle at least a portion of the waist of the wearer and form a portion of the waist opening. For embodiments having two pairs of side panels, the first pair of laterally opposing side panels, such as first and second side panels 62a, 62b, extend from the laterally opposing side edges of the containment assembly 22 in a first waist region, for example rear waist region 44 over the wearer's hips. The second pair of laterally opposing side panels, such as the third and fourth side panels 38a, 38b, extend from the laterally opposing side edges of the containment assembly 22 in the longitudinally opposing waist region, namely, the front waist region 46, of the diaper 20. As shown in FIG. 7, which illustrates a partial view of the diaper 20 in a pull-on configuration, the pairs of side panels 38 and 62 may be fastened together forming a closed waist circumference 71 and a pair of closed leg openings 72. For an embodiment having one pair of side panels, the side panels may be attached to and extend from the laterally opposing side edges 50 of the containment assembly 22 in a first waist region over the wearer's hips, to the corresponding side edges 50 of the containment assembly 22 in the longitudinally opposing waist region of the diaper where the side panels are fastened to the containment assembly 22 to form a closed waist circumference 71. As illustrated in FIG. 8, for example, the first side panel 62a extends past the side edge 50 and is attached to a portion of the front waist region 46 to form a closed waist circumference 71.

In one embodiment, at least one pair of side panels, such as the first and second side panels 62a, 62b, may comprise releasable bonds in addition to one or more engaging components positioned laterally outward of the engaging component 202. FIG. 7 illustrates a portion of the diaper 20 having releasable bonds 35 in accordance with one non-limiting embodiment. The releasable bonds help to distribute the forces applied to the side panel across substantially the full longitudinal length of the side panel thereby reducing the likelihood of unwanted effects, such as side panel roping and skin marking. The diaper 20 may have any number of releasable bonds 35. The embodiment in FIG. 7 shows two circular releasable bonds 35. Other embodiments may have more or less releasable bonds in similar or different shapes. For example, in some embodiments, the releasable bond 35 may be arranged in a strip or a series of strips. The releasable bonds 35 may be, for example, semi-permanent bonds that help maintain the position of a side panel relative to other components and also help maintain the diaper in a pull-on configuration until the releasable bonds are released. The releasable bonds 35 may be formed by any bonding means as is known in the art, including, but not limited to, those bonding means described above with regard to the side panels. These releasable bonds 35 may be formed such that they peel apart without damage to the substrates between which the bonds were formed. Alternatively the releasable bonds 35 may be formed such that when separated they create controlled localized damage to one or both of the substrates with which the bonds were formed.

FIG. 8 illustrates a side panel comprising a force distribution portion 80 in accordance with one non-limiting embodiment. As shown, the force distribution portion 80 may be a portion of the first side panel 62a, for example. The force distribution portion 80 may be a relatively stiff and/or non-extensible portion of the first side panel 62a that helps to distribute forces exerted on the fastening portion 62a of the side panel during use. By distributing the forces to the edges of the side panel, concentrated strain on the engaging component(s) 202 may be reduced or eliminated and the comfort of the user may be increased. The force distribution portion 80 may be implemented using various techniques. For example, the force distribution portion 80 may be a portion of relative stiffness in the first side panel 62. In some embodiments, the force distribution portion 80 may comprise a force distribution member 81 that may be attached to, integrated with, unitary with, or otherwise joined to the first side panel 62a. The force distribution member 81 may be manufactured from any suitable rigid, semi-rigid or non-extensible material, such as, for example, films, foams, structured nonwovens, etc.

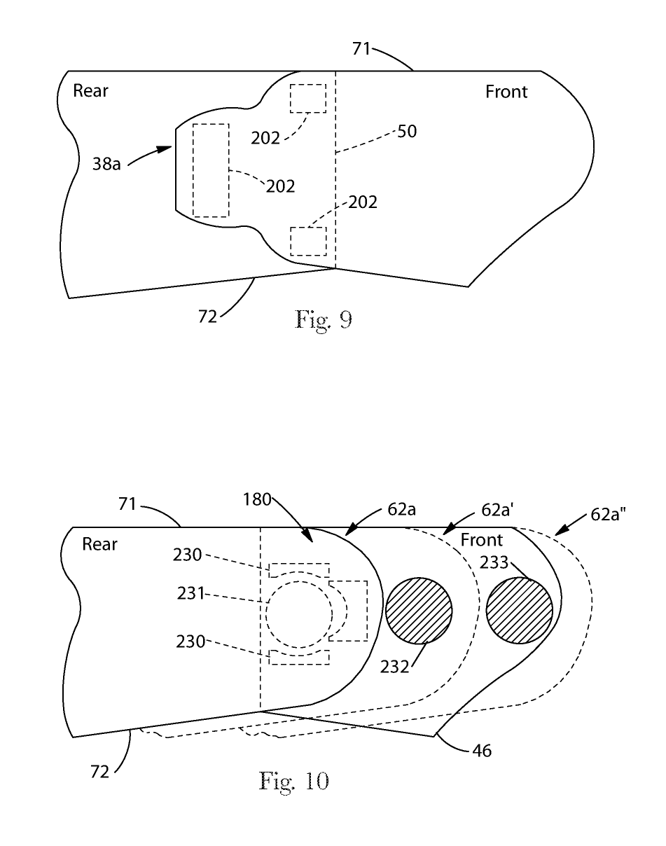

FIG. 9 illustrates an embodiment of the diaper comprising a single pair of panels, namely third and fourth panels 38a, 38b. As illustrated, the third side panel 38a extends past the side edge 50 and attach to a portion of the rear waist region 44 to form a closed waist circumference. Furthermore, the engaging component 202 may be attached to either the third side panel 38a or the rear waist region 44. In this embodiment, the specific formation of the multiple fastening components distributes the force across the longitudinal length of the side panel. The distal fastener 202 is disposed such that it can carry the forces associated with the central portion of the side panel. The pair of proximal fasteners 202 are disposed laterally inward of the distal fastener 202 and are disposed such that they can carry the forces associated with the longitudinally distal and longitudinally proximal portions of the side panel. Such a design distributes the forces associated with application and wear of the product substantially uniformly across the surface of the side panel thereby reducing potential for skin marking associated with the side panel. As is to be appreciated, releasable bonds and/or a force distribution portion may be implemented with this configuration.

In some embodiments, the close waist circumference may be adjustable to accommodate different sized wearers or otherwise change the comfort and/or fit of the diaper 20. FIG. 10 illustrates an embodiment of the diaper comprising an adjustable fastening system 180. The fastening system 180 allows for varying degrees of tightness (i.e., changing the waist hoop circumference) and may comprise complementary or interacting engaging and/or landing components in a variety of shapes and configurations. For example, a first closure 230 may be attached to the first side panel 62a and engaging components 231-233 may be position on the front waist region 46. While three engaging components 231-233 are illustrated, it is to be appreciated that any suitable number of complementary engaging components may be utilized. In the illustrated embodiment, when the first side panel 62a is secured proximate to the first engaging components 231 the closed waist circumference is relatively large. When the first side panel 62a′ is secured to the second complementary closure 232, the waist hoop circumference decreases compared to the first position, thereby increasing tightness. When the first side panel 62a″ is secured to the third engaging components 233, the waist hoop circumference is relatively small. While three positions of incrementable snugness is illustrated, it is to be appreciated that greater or fewer positions may be implemented in various embodiments.

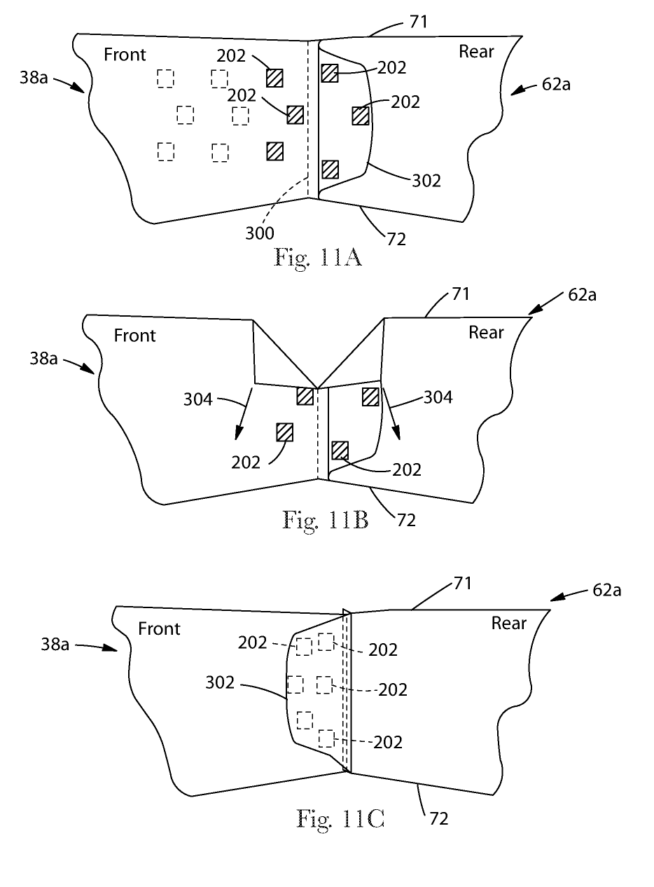

FIGS. 11A-11C illustrates a progression of a permanent seam configuration in accordance with one non-limiting embodiment. As shown in FIG. 11A, the third side panel 38a may be joined to, or be unitary with, the first side panel 62a at a permanent seam 300. The permanent seam 300 may be constructed in such a fashion to be frangible to allow a user to separate the two panels at or adjacent that seam. One of the panels, such as the first side panel 62a may comprise a flap 302 comprising engaging components 202. The flap 302 may be any suitable configuration, such as a tab, wing, or other foldable member. The other side panel, such as the third side panel 68a may also comprise engaging components 202. As illustrated, in some embodiments, the third side panel 68a may comprise a plurality of engaging components 202 to allow for size adjustment. In such an embodiment, the engaging components 202 may be engaged with a complementary landing, mating, component disposed on or forming a portion of the facing surface of the opposing side panel. Alternatively, a non-engagement layer or surface may be disposed between the engaging components 202 and the underlying side panel to maintain the fastener in a non-engaged state until the side seam is broken and the fastener is fastened to another portion of the diaper, a landing component 204. Further, while not illustrated, various embodiments may comprise various configurations of landing components. When the side panels are joined at the permanent seam 300, the diaper is in a “pull-on” configuration. As shown in FIG. 11B, the permanent seam 300 may be opened by pulling in the direction indicated by arrows 304. In various implantations, the permanent seam 300 may be opened either before the diaper is worn, or after the diaper has been placed about the wearer's hips. As shown in FIG. 11C, once the permanent seam 300 has been completely opened the engaging components 202 on the flap 202 may be fastened to the third side panel 38a, and in the illustrated embodiment, the engaging components 202 on the third side panel 38a may be fastened to the flap 302.

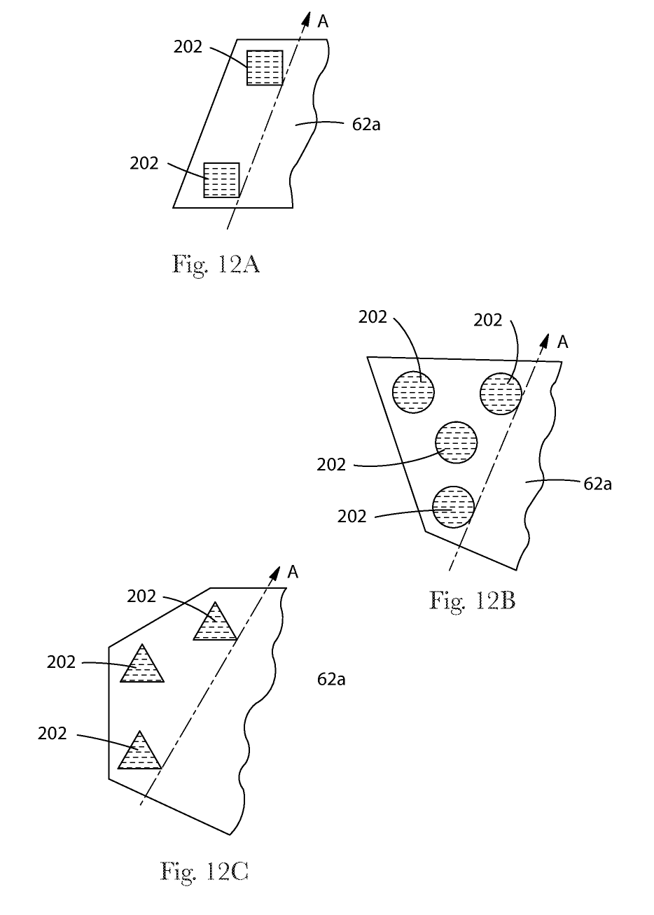

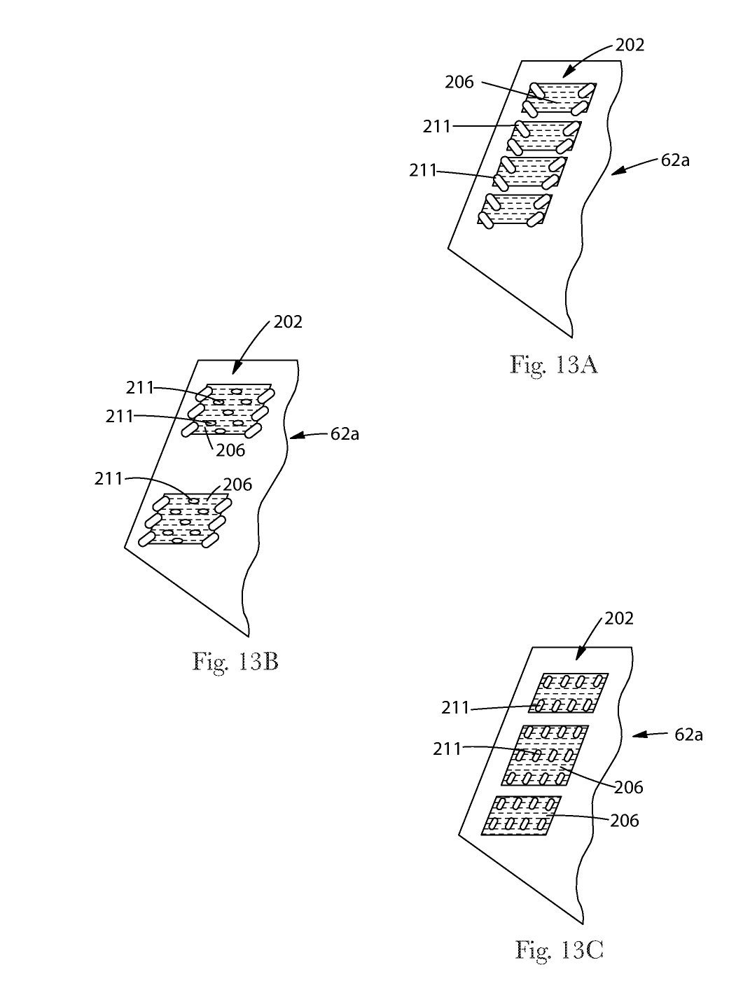

As shown in FIGS. 12A-C, the first and second engaging components 202 of the fastening system 200, may each comprise more than one engaging element. Further, the engaging elements 206 may be similar or different in size and/or shape. In some embodiments, the multiple engaging components 202 should be arranged to gain the benefits described above with regard to the single angled engaging components 202. The “closure member major axis A” in multi-component embodiments is drawn between the laterally inboardmost points of each engaging component 202. The closure member major axis A may be nonparallel to the longitudinal centerline 100 of the absorbent article. In some embodiments, the closure member major axis A may converge toward the longitudinal centerline 100 as it moves away from the lateral centerline 102 of the absorbent article toward the end edge 52 in the rear waist region 44 (when in a flat out configuration).

The various fastening elements, such as the engaging component 202 and/or the landing component 204, for example, may be formed integrally with a portion of the tab, a portion of the side panel and/or a portion of the containment assembly 22, or the fastening elements may be formed from separate members joined to the tab, side panel or absorbent assembly. For example, means for joining the fastening elements to the tab, containment assembly and/or the side panel include, but are not limited to adhesives, cohesives, heat, pressure, ultrasound, mechanical bonding or any combination of these or other known bonding means. For example, the engaging component 202 may be joined to the tab member, containment assembly 22 or side panel with an adhesive as well as a mechanical bond. Mechanical bonding, or fusion of some form may be used alone, or in combination with other bonding means. (The term “mechanical bond” as used herein, refers to bonds formed by means of pressure, ultrasound, heat, laser energy or any form of energy input which mechanically joins the elements.) In one embodiment an adhesive bond is used to join one or more of the engaging components 202 to the absorbent article, supplemented by a mechanical bond.

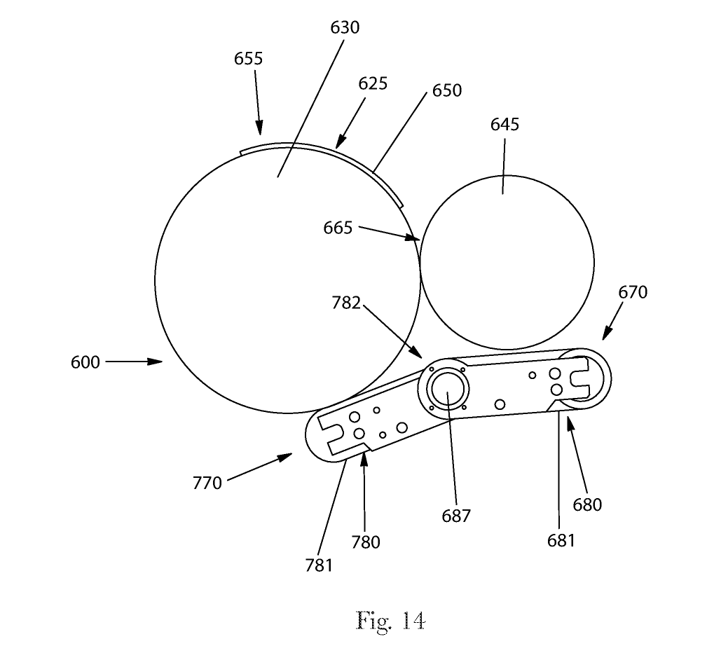

It has been found that if a mechanical bond is used to join the engaging component 202 to an element of the absorbent article, alone or in combination with other bonding means, the mechanical bond pattern may affect the strength of the bond between the engaging component 202 and the element to which it is joined, as well as the fastening characteristics of the engaging component 202. Mechanical bonding typically damages at least some of the engaging elements 206 of the engaging component 202, causing some change in the fastening performance of the component. Examples of bond patterns comprising various types of mechanical bonds 211 are shown in FIGS. 13A-C. The bonding patterns shown in FIGS. 13A-C are generally effective in securely joining the engaging component 202 with the element to which it is being bonded while minimizing the impact of the mechanical bonds 211 on the engaging elements 206, and thus, the engaging component's overall fastening performance. This type of bonding and fastening performance is important for products such as the diaper 20 in which the engaging elements 206 control the majority of the side panel when in use. The implication of this control of the side panel is that shear and peel forces provided by the movements of the wearer tend to separate the engaging component 202 from the side panel or tab to which it is joined. Such forces may also disengage the engaging component 202 from its complementary landing component 204.

In some embodiments, the mechanical bonds 211 may occupy less than about 20%, or less than about 10%, or less than about 7% of the area of the central region 218. This will assure that the mechanical bonds 211 do not damage too many engaging elements 206, and thus, will not significantly reduce the effectiveness of the engaging component 202 or the overall fastening system 200. The percentage of area occupied by the bonds is measured by examining a representative sample of the engaging component 202 under a microscope. The sample is viewed under the microscope from directly above the side from which the engaging elements 206 extend. The plan view area of each of the mechanical bonds 211 is measured. The sum of the areas of the bonds is divided by the area of the sample. The result is expressed as a percentage. This is the percentage of area occupied by the bonds.

Attorney Docket Nos. 11573P and 11575P filed on Jan. 20, 2010, naming Gary Dean LaVon as an inventor, generally discloses various acceptable product features that may be implemented in the embodiments described in the present application. Specifically, the above-described embodiments may, as appropriate, comprise a first fastening component, a second fastening component, a non-engagement zone, and a separation zone as described in Attorney Docket Nos. 11573P and 11575P Further, the embodiments as described above may, as appropriate, comprise on overlap seam and/or a flange seam as described in Attorney Docket Nos. 11573P and 11575P.

### Method of Making

In one embodiment provides a bifold assembly system for folding an article along a laterally extending fold line. The system may include a first vacuum conveyance, for example a vacuum drum, that has an outer surface for receiving discrete absorbent articles. The first vacuum conveyance may transport the discrete absorbent article in the machine direction. The article having a leading half comprising the front waist region and the front half of the crotch region, i.e. the portion of the crotch region disposed between the lateral centerline and the front waist region, and a trailing half comprising the rear waist region and the back half of the crotch region, i.e. the portion of the crotch region disposed between the lateral centerline and the back waist region. The system may also include a second vacuum conveyance, for example a second vacuum drum, disposed adjacent to the first vacuum conveyance. The second vacuum conveyance may be configured to receive at least a portion of the leading half of the article from the first vacuum conveyance. The second vacuum conveyance may be configured to apply a peel force such that at least part of the leading half of the absorbent article is transferred from the first vacuum conveyance to the second vacuum conveyance and held on the surface of the second vacuum conveyance. The system may further includes a third vacuum conveyance, for example a vacuum conveyor, disposed proximate the first and second vacuum conveyances. The third vacuum conveyance may include a drive mechanism for moving the surface of the third vacuum conveyance at a first speed, a second speed, in a first direction and in a second direction.

In one embodiment of the method of making, a continuous web comprising a plurality of interconnected backsheets is combined with a series of laterally opposing pairs of discrete side panels. The side panels may comprise a material that is elastically extensible and may additionally comprise a refastenable fastening component disposed on a surface of the side panel. The continuous web of interconnected backsheets is subsequently joined with a continuous web comprising a plurality of interconnected topsheets. Discrete absorbent cores are disposed between the continuous backsheet web and the continuous topsheet web thereby forming a plurality of interconnected absorbent containment assemblies. The web is severed to form a plurality of discrete absorbent articles. A discrete absorbent article is placed on the outer surface of a first vacuum conveyance 630. Alternatively, the continuous web of interconnected absorbent articles may be placed on the first vacuum conveyance prior to severing the web into discrete absorbent articles. The first vacuum conveyance 630 may be in the form of a vacuum drum as shown in FIG. 14 or alternatively in the form of a movable foraminous vacuum conveyor belt configured in an endless loop. The article is transported along the surface of the first vacuum conveyance 630 in a direction toward a second vacuum conveyance 645. The second vacuum conveyance 645 may be in the form of a vacuum drum as shown in FIG. 14 or alternatively in the form of a movable foraminous vacuum conveyor belt configured in an endless loop. A portion of the leading half of the absorbent article comprising the front waist region 46 and the front half of the crotch region 48 is transferred from the first vacuum conveyance 630 to the second vacuum conveyance 645. Bifold clamps (not shown) hold the crotch region 48 at or adjacent the lateral centerline 102 in contact with the first vacuum conveyance 630 thereby helping to maintain the trailing half of the article comprising the back half of the crotch region 48 and the back waist region 44 in contact with the surface of the first vacuum conveyance 630. The surfaces of the first vacuum conveyance 630 and second vacuum conveyance 645 downstream of the transfer point are moving in directions that are divergent from one another. The leading half of the article is transported along the surface of the second vacuum conveyance until it is transferred to a third vacuum conveyence 670. The surfaces of the third vacuum conveyance and the second vacuum conveyance are convergent. The bifold clamp not only helps retain the crotch region in contact with the first vacuum conveyance but it also helps retain tension in the article as it is bifolded. The leading half of the article travels along the surface of the third vacuum conveyance as the trailing half of the article travels along the surface of the first vacuum conveyance thereby forming a bifolded absorbent article. At this point the surface of the third vacuum conveyance and the surface of the first vacuum conveyance are moving in a convergent direction. U.S. Pat. Publ. No. 2009/0098995A1, entitled System for Bifolding an Absorbent Article, generally discloses various acceptable embodiments for carrying out the process as described above and as described in the present application's FIG. 35. More specifically, the first vacuum conveyance (folding drum), second vacuum conveyance (peel roll), third vacuum conveyance (bifold conveyor) as disclosed in U.S. Pat. Publ. No. 2009/0098995A1 may be used in the process of the present application. Further, U.S. Pat. Publ. No. 2008/0083489 and U.S. Pat. No. 5,779,831 generally disclose various acceptable embodiments for carrying out an alternative process (versus the ones describe and referenced above). More specifically, the processing wheel, processing station (folding device), gripper and sealer (side panel folding and seaming) as disclosed in U.S. Pat. Publ. No. 2008/0083489 and U.S. Pat. No. 5,779,831 may be used in the process of the present application. Still further, Attorney Docket Nos. 11573P and 11575P, filed on Jan. 20, 2010, naming Gary Dean LaVon as an inventor, also generally discloses various acceptable embodiments for carrying out an alternative process (versus the ones describe and referenced above).

The trailing half of the absorbent article comprising the back waist region may comprise side panels having refastenable fastening components disposed thereon. The side panels are held in place by vacuum created by the first vacuum conveyance. Once the article has been bifolded, a portion of the side panel, such as the portion comprising the refastenable fastening components may be folded laterally inward to engage the fastening components disposed in the back waist region with the front waist region of the absorbent article. The side panels may be folded laterally inward by means of a fourth vacuum conveyance such as a vacuum twist belt or alternatively the side panels may be folded laterally inward by a folding board or other means known in the art. In an alternative embodiment the side panels may be folded by a folding mechanism disposed on a potion of the first vacuum conveyance.

Once folded one or more releasable bonds may be formed joining a portion of the front waist region to a portion of the rear waist region. The bonds may be formed by any means previously described herein

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description are, in relevant part, incorporated herein by reference in their entirety. The citation of any document is not to be construed as an admission that it is prior art with respect to the present disclosure. To the extent that any meaning or definition of a term in the present disclosure conflicts with any meaning or definition of the term in a document incorporated by reference, the meaning or definition assigned to the term in the present disclosure document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. An absorbent article comprising:
a longitudinal centerline and a lateral centerline;
a containment assembly comprising a topsheet and a backsheet;
a front waist region;
a rear waist region;
a crotch region disposed between the front waist region and the rear waist region;
a first elastically extensible side panel extending beyond a first side edge of the containment assembly in the rear waist region;
a second elastically extensible side panel extending beyond a second side edge of the containment assembly in the rear waist region;
a third elastically extensible side panel extending beyond a first side edge of the containment assembly in the front waist region;
a fourth elastically extensible side panel extending beyond a second side edge of the containment assembly in the front waist region;
a refastenable fastening system, the refastenable fastening system comprising:
a first engaging component permanently joined to a garment facing surface of the first side panel, the first engaging component comprising hooks;
a second engaging component permanently joined to a garment facing surface of the second side panel, the second engaging component comprising hooks;
a first landing component permanently joined to or integral with the wearer facing surface of the third side panel, the first landing component comprising fibers;
a second landing component permanently joined to or integral with a wearer facing surface of the fourth side panel, the second landing component comprising fibers;
wherein the first and second side panels each comprise a permanent side seam adjacent to the engaging components, the permanent side seams running parallel to the longitudinal centerline; and
wherein the first and second engaging components are refastenably joined to the first and second landing components, respectively, such that the absorbent article is in a pull-on configuration.
